(19) United States
(12) Patent Application Publication
(10) Pub. No.: US 2020/0316103 A1
(43) Pub. Date: Oct. 8, 2020

(54) MEDICINAL COMPOSITION

(71) Applicant: Eva Dowlen, Phoenix, AZ (US)

(72) Inventor: Eva Dowlen, Phoenix, AZ (US)

(21) Appl. No.: 16/377,229

(22) Filed: Apr. 7, 2019

(51) Int. Cl. A61K 31/714; A61K 36/185; A61K 36/28

(52) U.S. Cl. CPC A61K 31/714; A61K 36/28; A61K 36/185

(57) ABSTRACT

A medicinal composition for preventing, ameliorating, or at least reducing the pain intensity associated with a headache is disclosed. The composition comprises a therapeutic amount of a plurality of Humulus lupulus, Tanacetum parthenium, and Vitamin B12.

### TECHNICAL FIELD

The embodiments generally relate to therapeutic compositions which aid in relief from pain associated with primary and secondary headaches.

### BACKGROUND

There are more than 200 types of headaches which can be categorized as primary headaches or secondary headaches. Primary headaches are benign; however, it may cause the inability to perform daily tasks. Secondary headaches are caused by an underlying disease, such as an infection, head injury, vascular disorder, or tumors. Over 90% of all headaches are primary headaches such as a migraine, cluster headache, migraines with aura, ocular migraines, or stress headaches to name a few.

In the United States, over 40 million people suffer from pain associated with daily migraines and headaches. The daily discomfort can be debilitating and cause significant physical and emotional strain. There are many causes of headaches which require various treatments or preventative protocols. Lifestyle and behavioral changes are often recommended to help the patient avoid specific triggers. Preventative medicines therapies are also available including beta blockers and NSAID's among others depending on the associated symptoms at the time of presentation.

In recent years, the use of therapies previously thought to be safe is often discouraged. In particular, certain NSAID's are associated with various long-term health risks. To reduce the risk of long-term side effects, many individuals have begun to employ homeopathic remedies to prevent or at least reduce the pain or associated with the headaches. Many homeopathic remedies include lifestyle changes including regular exercise, diet changes, stress relief, and the implementation of herbal supplements or medicaments.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments described herein provide for a medicinal composition for preventing, ameliorating, or at least reducing the pain intensity associated with a headache. The composition comprises a therapeutic amount of a plurality of Humulus lupulus, Tanacetum parthenium, and Vitamin B12.

The composition may be utilized to effectively prevent or ameliorate various types of headaches including migraines, cluster headaches, tension headaches, migraines with aura, ocular migraines, and stress headaches in addition to other types of headaches known in medicine. The composition is administered orally via the ingestion of a plurality of capsules at least twice each day as a prophylactic to prevent the onset of a headache. Upon the onset of headache symptoms, the composition may be ingested to ameliorate or at least reduce the pain associated with the headache.

In one aspect, the composition is provided in vegetable capsules using vegetable cellulose.

In one aspect, the composition is further comprised of a coloring to improve the visual aesthetic of the composition.

In one aspect, the coloring is Titanium Dioxide.

In one aspect, the therapeutic amount of the Humulus lupulus and the Tanacetum parthenium is about 558.5 milligrams per capsule.

In one aspect, the therapeutic amount of the Vitamin B12 is about 6 micrograms per capsule.

In one aspect, the serving size is two capsules which may be taken twice per day.

The embodiments also provide for a method for preventing, ameliorating, or at least reducing the pain intensity associated with a headache via administering a therapeutically effective amount of a composition. At least four capsules are administered each day as a prophylactic, and/or upon the onset of headache symptoms.

### DETAILED DESCRIPTION

The specific details of the single embodiment or variety of embodiments described herein are to the described composition. Any specific details of the embodiments are used for demonstration purposes only and not unnecessary limitations or inferences are to be understood therefrom.

Before describing in detail exemplary embodiments, it is noted that the embodiments reside primarily in a composition to provide relief from pain associated with headaches and similar pain in the head and neck. The active ingredients of the composition are comprised of ingredients known to treat, ameliorate, or at least reduce pain intensity associated with a headache in a host suffering therefrom when orally administered.

The term “therapeutically effective amount” should be construed herein to include a nontoxic but sufficient amount of the agent or ingredient to provide the desired therapeutic effect. The exact amount may vary from subject to subject, depending on the age, weight, and general condition of the subject, the severity of the condition being treated, and the like. Thus, it is not possible to specify an exact “effective amount.” However, an appropriate “effective amount” in any individual case may be determined by one of ordinary skill in the art using only routine experimentation.

Throughout this application, the term “about” is used to indicate that a value includes the standard deviation of error for the composition and deployment thereof to determine the value. In an embodiment, this deviation is ±10% of a given value.

The term “headache” should be construed to also include, without limitation, a tension headache, a migraine, a sinus headache, a cluster headache, a rebound headache or any other type of headache known in the arts including cranial aches and pain and neck pain. Headaches are a common problem and are usually not a sign of a severe medical condition. Headaches can be caused by a number of different factors such as stress/anxiety, alcohol, atmosphere, lighting, sitting at a computer for long periods, allergies, eye strain, low blood sugar, grinding teeth, poor posture, sleep loss, etc. It is contemplated that the composition is utilized as a means for reducing stress to prevent headaches from occurring.

The term “pharmaceutically acceptable carrier” or “pharmaceutically acceptable vehicle” or “excipient” should be construed herein to include materials that are suitable for ingestible administration and are not biologically or otherwise undesirable, i.e., that may be administered to an individual along with an active agent without causing any undesirable biological effects.

In some embodiments, the composition may be administered topically using a pharmaceutically acceptable carrier, or excipient wherein the composition is applied directly to or proximal the cephalic region of the host suffering from symptoms associated with a headache.

The composition includes therapeutically effective amounts of Humulus lupulus (hops), Tanacetum parthenium (feverfew), and Vitamin 12. In one example, the therapeutically effective amounts are encapsulated and may be allocated to a plurality of capsules for a single serving. Each capsule may be provided with an amount of the composition. In one example, the user orally ingests a prescribed number of capsules depending on the severity of their symptoms, body composition, or tolerance.

In some embodiments, Vitamin B12 is provided as cyanocobalamin, a manufactured form of the Vitamin B12. One skilled in the arts will understand that various vitamers of Vitamin B12 may be utilized in the composition.

In some embodiments, the composition is provided having a capsule containing about 558.5 milligrams Humulus lupulus and Tanacetum parthenium, and about 6 micrograms Vitamin B12. A serving (i.e., a single dose) contains about 1117 milligrams of Humulus lupulus and Tanacetum parthenium and 12 micrograms of Vitamin B12. Table 1 below illustrates an exemplary composition according to the embodiments.

TABLE 1

| Active Ingredient | Amount Per Serving | |
|---|---|---|
| Vitamin B12 | 12 | micrograms |
| Humulus lupulus | 1117 | milligrams |
| Tanacetum parthenium | | |

In some embodiments, the Humulus lupulus is provided in powdered form from the whole Humulus lupulus flower. One skilled in the arts will appreciate that the Humulus lupulus may be provided in other forms such as a tincture, distillation, oil, extract, or other means known in the arts.

In some embodiments, Tanacetum parthenium is provided in powdered form from the whole Humulus lupulus flower. One skilled in the arts will appreciate that the Humulus lupulus may be provided in other forms such as a tincture, distillation, oil, extract, or other means known in the arts.

In some embodiments, coloring agents may be utilized to provide a color to the powder for aesthetic or functional purposes. In one example, the coloring agent is Titanium dioxide. However, similar coloring agents known in the arts may be employed.

In some embodiments, the composition is provided without any filling agents, binding agents, artificial ingredients, gluten, wheat, dairy, or GMO's.

The composition is formulated having medicinal properties including relief from stress, nausea, premenstrual syndrome, high blood pressure, hormonal imbalances, local and systemic inflammation, light sensitivity, noise sensitivity, and insomnia which often accompany or are triggers for headaches.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification shall be construed to constitute a complete written description of all combinations and subcombinations of the embodiments described herein, and of the manner and process of making and using them and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A medicinal composition for preventing, ameliorating, or at least reducing the pain intensity associated with a headache, the composition comprising:
a therapeutic amount of a plurality of active ingredients comprising Humulus lupulus, Tanacetum parthenium, and Vitamin B12.

2. The composition of claim 1, wherein the composition is provided in a capsule.

3. The composition of claim 2, wherein the capsule is comprised of a vegetable cellulose.

4. The composition of claim 3, further comprising a coloring.

5. The composition of claim 4, wherein the coloring is Titanium Dioxide.

6. The composition of claim 1, wherein the therapeutic amount of the Humulus lupulus and the Tanacetum parthenium is about 558.5 milligrams per capsule.

7. The composition of claim 1, wherein the therapeutic amount of the Vitamin B12 is about 6 micrograms per capsule.

8. The composition of claim 1, wherein Vitamin B12 is provided as cyanocobalamin.

9. A medicinal composition for preventing, ameliorating, or at least reducing the pain intensity associated with a headache, the composition comprising:
a therapeutic amount of a plurality of active ingredients provided in a capsule, the capsule comprised of about 558.5 milligrams Humulus lupulus and Tanacetum parthenium, and about 6 micrograms Vitamin B12, the capsule further comprising a coloring agent.

10. The composition of claim 9, wherein the capsule is comprised of a vegetable cellulose.

11. The composition of claim 9, wherein the coloring is Titanium Dioxide.

12. The composition of claim 9, wherein the composition is administered at least two times per day to prevent the onset of a headache.

13. The composition of claim 12, wherein the composition is further administered upon the onset of a headache.

14. The composition of claim 9, wherein the Humulus lupulus is provided as a powder of a Humulus lupulus flower.

15. The composition of claim 14, wherein the Tanacetum parthenium is provided as a powder of a Tanacetum parthenium flower.

16. A method for preventing, ameliorating, or at least reducing the pain intensity associated with a headache via a therapeutically effective amount of a composition, the method comprising the steps of:
administering a therapeutic amount of a plurality of active ingredients provided in a capsule, the capsule comprised of about 558.5 milligrams Humulus lupulus and Tanacetum parthenium, and about 6 micrograms Vitamin B12, the capsule further comprising a coloring agent, the capsule administered at least 4 times per day.

17. The method of claim 16, wherein the Humulus lupulus is provided as a powder of a Humulus lupulus flower.

18. The method of claim 16, wherein the Tanacetum parthenium is provided as a powder of a Tanacetum parthenium flower.

19. The method of claim 16, wherein the composition is administered upon the onset of a headache.

20. The method of claim 16, wherein the coloring is Titanium Dioxide.
